(19) United States
(12) Patent Application Publication
(10) Pub. No.: US 2022/0211103 A1
(43) Pub. Date: Jul. 7, 2022

(54) PORTABLE VAPORIZERS WITH HEALING CRYSTAL ATTACHMENTS

(71) Applicant: Aroon Attai, Manassas, VA (US)

(72) Inventor: Aroon Attai, Manassas, VA (US)

(21) Appl. No.: 17/248,064

(22) Filed: Jan. 7, 2021

(51) Int. Cl. A24F 40/40; A44C 17/02; A61M 15/06

(52) U.S. Cl. CPC A24F 40/40; A61M 15/06; A44C 17/02

(57) ABSTRACT

The present disclosure provides a portable vaporizer including a healing crystal attached thereto. In embodiments, the healing crystal may be attached to the portable vaporizer via one or more magnets. In embodiments, the healing crystal may be attached to the portable vaporizer via a screw attachment. In embodiments, the portable vaporizer may be an e-cigarette. In embodiments, the crystal may be easily and conveniently attached and detached from the portable vaporizer.

### BACKGROUND OF THE DISCLOSURE

### Field of the Invention

This disclosure is directed to portable vaporizers coupled with healing crystals, gemstones, and other precious crystals, etc., that promote healing and good health.

### Description of the Related Art

Healing crystals have been used for thousands of years promoting positive energy and wellness. Today, they are an established form of alternative medicine. There are no known applications of such crystals to portable vaporizers. Providing the healing and wellness benefits of healing crystals, and the like, to a portable vaporizer or an e-cigarette product would provide new alternative medicine healing capabilities for the mind and body. There are a number of different known types of healing crystals, each providing their own healing abilities for the mind and body. Such crystals promote good health and provide physical and emotional benefits. Attaching them with ease to a portable vaporizer could enhance a user's experience while, for example, vaping.

### SUMMARY OF THE INVENTION

The present disclosure provides a portable vaporizer including a healing crystal attached. In embodiments, the healing crystal may be attached to the portable vaporizer via one or more magnets. In embodiments, the healing crystal may be attached to the portable vaporizer via a screw attachment. In embodiments, the portable vaporizer may be an e-cigarette. In embodiments, the crystal may be easily and conveniently attached and detached from the portable vaporizer.

Being able to add the interchangeable functionally of healing crystals/stones to a vaporizer provides a customizable experience for a user to modify their vaping experience whenever the user pleases.

Other features and aspects will be apparent from the following detailed description, the drawings, and the claims.

### BRIEF DESCRIPTION OF THE DRAWINGS

FIG. 1 shows one embodiment of a portable vaporizer with healing crystal attachment.

FIG. 2 shows another embodiment of a portable vaporizer with healing crystal attachment.

FIG. 3 shows another embodiment of a portable vaporizer with healing crystal attachments.

FIG. 4 shows another embodiment of a portable vaporizer with healing crystal attachment of the disclosure.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

Attaching healing crystals to a vaping product provides benefits including enhancing the experience for the user and providing alternative medicine healing capabilities. For example, healing crystals can enhance a meditation experience which is known to provide significant health benefits such as the reduction of stress, anxiety, etc. Healing crystals are also used to attract positive energy, love, and abundance.

Each crystal has a different intention/purpose. Accordingly, the interchangeability of healing crystals on a portable vaporizer can further benefit a user by promoting positivity and wellness while simultaneously being able to swap out crystals for different purposes.

The crystals depend on the needs of a user. Regardless of what the user is using their portable vaporizer for, the option of modifying the experience via interchangeable mood-enhancing crystals is a beneficial function that is currently not available in the portable vaporizer market. Not only does this provide ease and functionality of being able to conveniently modify vaping hardware but also provides an aesthetically pleasing, alternative health benefit, customizable feature for the user. On top of, and in addition to, the healing elements of crystals, precious stones can also be added such as diamonds, sapphires, and emeralds, etc., to provide a luxurious and unique enhancement for modern vaporizers.

The crystals include but are not limited to: Alexandrite, Amazonite, amber, Amethyst, Aquamarine, Aventurine, Azurite, Bloodstone, Calcite, Carnelian, Celestite, Chrysoprase, Citrine, Diamond, Dioptase, Emerald, Fluorite, Garnet, Gold, Hematite, Herkimer Diamond, Jade, Jasper, Kunzite Pink, Lapis Lazuli, Lepidolite, Malachite, Moonstone, Obsidian, Onyx, Opal, Peridot, Pyrite, Quartz Crystal, Rhodochrosite, Rhodonite, Rose Quartz, Ruby, Rutilated Quartz, Smoky Quartz, Sugilite, Sodalite, Sapphire, Silver, tiger Eye, Topaz, Tourmaline, Turquoise, Zircon.

Each of the above have a variety of known healing properties.

The healing crystals can take a variety of forms including the form of crystals and may be crushed, altered, fine fibers, thin pellet disks or half/whole crystals. As such, the crystal can include any combination of a crystal, a mineral, an orgonite, a stone, a gemstone, a rock, a crystal cluster, a mineral, a fossil, and/or a rune stone.

The crystal can have a diameter or cross section ranging from about ⅛ inch to about 5 inches. A preferred range would be about ¼ inch to about 2 inches or about ¼ inch to about 1 inch in a cross section or a diameter.

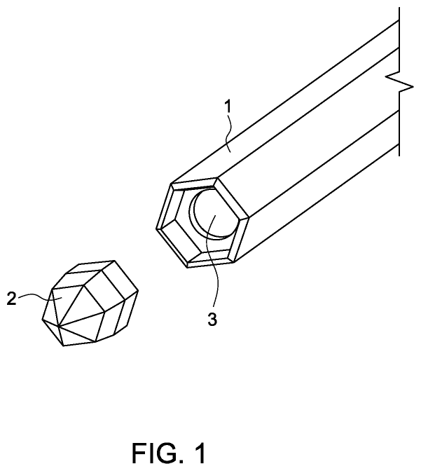

FIG. 1 shows one embodiment of a portable vaporizer with healing crystal attachment. FIG. 1 shows portable vaporizer 1, healing crystal 2, and one portion of magnetic attachment 3.

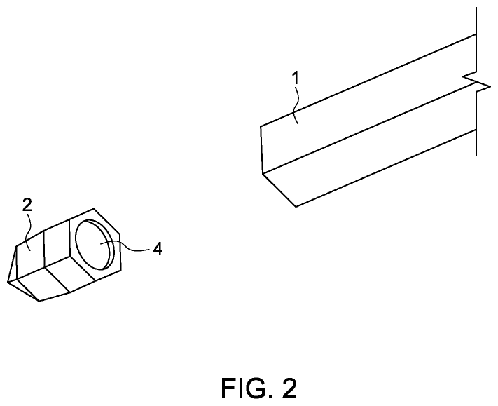

FIG. 2 shows another embodiment of a portable vaporizer with healing crystal attachment. FIG. 2 shows portable vaporizer 1, healing crystal 2, and one portion of magnetic attachment 4.

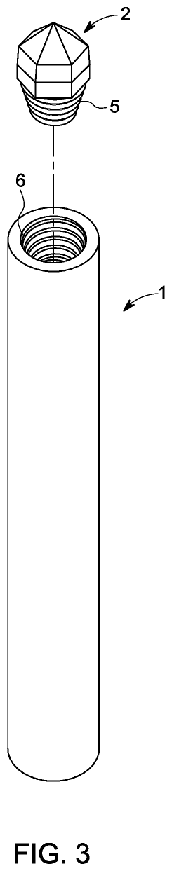

FIG. 3 shows another embodiment of a portable vaporizer with healing crystal attachments. FIG. 3 shows vaporizer 1, healing crystal 2 with screw base attachment 5, and tapped hole 6 in vaporizer 1.

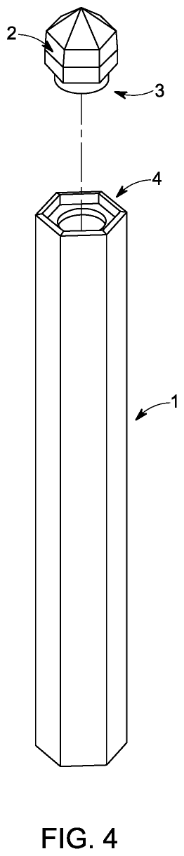

FIG. 4 shows another embodiment of a portable vaporizer with healing crystal attachment of the disclosure. FIG. 4 shows a hexagonal shaped vaporizer and crystal. FIG. 4 shows hexagonal shaped vaporizer 1, hexagonal crystal 2, magnet 3, and magnetic insert (with light) 4.

It is noted that a small light may be included, for example, at or near the place of attachment of the crystal to the vaporizer. In embodiments the light may be an LED light. In FIG. 4 the light is coupled with the magnetic insert 4 on the vaporizer. The light may be battery operated and of various colors.

As used herein, a vaporizer, sometimes known as a vape, includes devices used to vaporize substances for inhalation. Plant substances can be used, commonly cannabis, tobacco, or other herbs or blends of essential oil. However, they can also be filled with a combination propylene glycol, glycerin, and drugs such as nicotine (e.g., extract from tobacco) or tetrahydrocannabinol as a liquid solution.

Vaporizers may contain various forms of extraction chambers including straight bore, venturi, or sequential venturi, and are made of materials such as metal or glass. The extracted vapor may be collected in an inflatable bag or inhaled directly through a hose or pipe. When used properly, cooler temperatures due to lack of combustion may result in significantly more efficient extraction of the ingredients. Hence, the irritating and harmful effects of smoking are heavily reduced, as is its secondhand smoke. See Vaporizer (inhalation device), Wikipedia, The Free Encyclopedia, Date of last revision: 10 Dec. 2020; Date retrieved: 11 Dec. 2020, herein incorporated by reference in its entirety.

An electronic cigarette is a handheld battery-powered vaporizer that may simulate smoking by providing some of the behavioral aspects of smoking, including the hand-to-mouth action of smoking, but without combusting tobacco. Using an e-cigarette is known as vaping. Instead of cigarette smoke, the user inhales an aerosol, commonly called vapor. E-cigarettes typically have a heating element that atomizes a liquid solution called e-liquid. E-cigarettes may be activated by taking a puff; others turn on manually by pressing a button. Some e-cigarettes look like traditional cigarettes, but they come in many variations. See Id.

Attaching the crystal to the portable vaporizer may be done in a number of different ways. The crystal may be attached, for example, by magnets. In embodiments, a magnet of one polarity may be attached to a crystal and a magnet of opposite polarity may be attached to a vaporizer. For example, the magnets may be attached using a glue or a cement-based product, but other means of attachment known in the art for attaching small magnets to products and devices are also included. The crystal may then be magnetically attached and detached to the vaporizer. Magnetic attachments provide the feature of the user being able to easily swap out various crystals on a vaporizer.

Other attachment mechanisms may include attaching a crystal to a screw and attaching the crystal via a tapped hole in the vaporizer. Crystals can also be directly attached to a vaporizer by glue or a cement-based product.

While this disclosure includes specific examples, it will be apparent after an understanding of the disclosure of this application has been attained that various changes in form and details may be made in these examples without departing from the spirit and scope of the claims and their equivalents.

1. A portable vaporizer comprising:
a healing crystal attached thereto.

2. The portable vaporizer of claim 1, wherein the healing crystal comprises: Alexandrite, Amazonite, amber, Amethyst, Aquamarine, Aventurine, Azurite, Bloodstone, Calcite, Carnelian, Celestite, Chrysoprase, Citrine, Diamond, Dioptase, Emerald, Fluorite, Garnet, Gold, Hematite, Herkimer Diamond, Jade, Jasper, Kunzite Pink, Lapis Lazuli, Lepidolite, Malachite, Moonstone, Obsidian, Onyx, Opal, Peridot, Pyrite, Quartz Crystal, Rhodochrosite, Rhodonite, Rose Quartz, Ruby, Rutilated Quartz, Smoky Quartz, Sugilite, Sodalite, Sapphire, Silver, tiger Eye, Topaz, Tourmaline, Turquoise, and/or Zircon.

3. The portable vaporizer of claim 1, wherein the crystal is attached to the portable vaporizer via one or more magnets.

4. The portable vaporizer of claim 1, wherein the crystal is attached to the portable vaporizer via a screw attachment.

5. The portable vaporizer of claim 1, wherein the portable vaporizer is an e-cigarette.

6. The portable vaporizer of claim 1, wherein the crystal can be attached and detached from the portable vaporizer.

7. The portable vaporizer of claim 6, wherein the crystal is attached via one or more magnets.

8. The portable vaporizer of claim 1, wherein the crystal has a diameter or cross section ranging from about ⅛ inch to about 5 inches.

9. The portable vaporizer of claim 1, wherein the crystal has a diameter or cross section ranging from about ¼ inch to about 2 inches.

10. The portable vaporizer of claim 1, wherein the crystal has a diameter or cross section ranging from about ¼ inch to about 1 inch.

11. The portable vaporizer of claim 1, further comprising a gemstone.

12. The portable vaporizer of claim 11, wherein the gemstone comprises diamond, Sapphire, and/or Emerald.
